(19) United States
(12) Patent Application Publication
(10) Pub. No.: US 2024/0181496 A1
(43) Pub. Date: Jun. 6, 2024

(54) ELECTROACOUSTIC TRANSDUCER

(71) Applicant: Richard Wolf GmbH, Knittlingen (DE)

(72) Inventors: Edgar BAUER, Kraichtal (DE); Werner KRAUSS, Knittlingen (DE); Roland GAMER, Graben-Neudorf (DE)

(21) Appl. No.: 18/554,015

(22) PCT Filed: Apr. 7, 2022
(86) PCT No.: PCT/DE2022/200070

§ 371 Date: Oct. 5, 2023

(30) Foreign Application Priority Data
Apr. 9, 2021 (DE) 10 2021 203 544.2

(51) Int. Cl. B06B 1/06; A61B 17/225; B29C 45/63; G10K 15/04

(52) U.S. Cl. CPC B06B 1/0607; B29C 45/63; G10K 15/043; A61B 17/225; B06B 2201/55

(57) ABSTRACT

The electroacoustic transducer (1) is configured for the generation of shock waves for the treatment of the human or animal body. The electroacoustic transducer (1) includes piezoelectric elements (5), which are arranged within a bearer (4) in a housing (2, 3). Between the piezoelectric elements (5) and the housing (2, 3) a free space is formed, which is in-filled with a casting compound (10).

### CROSS REFERENCE TO RELATED APPLICATIONS

This application is a United States National Phase Application of International Application PCT/DE2022/200070, filed Apr. 7, 2022, and claims the benefit of priority under 35 U.S.C. § 119 of German Application DE 10 2021 203 544.2, filed Apr. 9, 2021, the entire contents of which are incorporated herein by reference.

### TECHNICAL FIELD

The invention relates to an electroacoustic transducer for the generation of shock waves, for the treatment of the human or animal body.

### BACKGROUND

Electroacoustic transducers can operate in accordance with different physical principles. The present invention relates to such electroacoustic transducers for the generation of shock waves, for the treatment of the human and animal body, which are constructed at least with the use of piezoelectric elements. Various designs of such transducers contribute to the prior art. There are focussed, linearly-focussed, and planar transducers, together with hybrid forms, which contribute to the prior art.

From DE 33 198 71 A1 a piezoelectric transducer for the destruction of concretions inside the body is of known art, in which the piezoelectric elements are arranged on a bearer in the form of a hemispherical shell; these are electrically connected and in-filled with a casting resin as a casting compound. By virtue of the arrangement of the elements on a spherical shell, the transducer is self-focussing. The interior of the transducer is in-filled with a liquid, and can be coupled to the body to be treated by way of a membrane. The casting resin must protect the piezoelectric elements from the liquid, which is problematical.

A similarly constructed electroacoustic transducer is of known art from DE 195 43 741 C1, in which the plastic casting compound is protected by a metal foil as a moisture barrier. Both embodiments have in common that the piezoelectric elements usually have to be attached to the bearer dome and electrically connected to each other by laborious manual work, after which the casting resin is in-filled, which requires a special tool that forms the part of the mould facing towards the coupling face. Here a separate tool is required for each transducer shape and size, which is time-consuming and expensive.

DE 10 2010 055 836 B4 contributes to the prior art with the construction in multiple layers of a self-focussing electroacoustic transducer that is constructed with piezoelectric elements. Here the piezoelectric elements are arranged in bearers that have a multiplicity of through-passage holes, in each of which a piezoelectric element is gripped and held. In this form of construction, too, a backing or supporting body can be provided on the rear side to ensure the mechanical hold of the transducer, but here, too, the composite is created by a casting compound that is in-filled using a special tool arranged on the coupling face. The tool must remain in position until the casting compound has hardened sufficiently.

### SUMMARY

Against this background, the invention as filed is based on the object of designing an electroacoustic transducer of the aforementioned type for the generation of shock waves for the treatment of the human or animal body, that is to say, one that is constructed using piezoelectric elements such that its manufacture is simplified. Furthermore, a method for the manufacture of such an electroacoustic transducer is to be provided.

The part of this object relating to the device is achieved by an electroacoustic transducer with the features specified. A method for the manufacture of such a transducer is characterized by the features specified in the drawings and disclosure. Advantageous configurations ensue from the following description, and the figures.

The electroacoustic transducer in accordance with the invention for the generation of shock waves for the treatment of the human or animal body is equipped with piezoelectric elements, which are arranged in at least one bearer within a housing. The free space formed between the piezoelectric elements and the housing is in-filled with a casting compound.

The basic concept of the present invention is that of the replacement of the otherwise expensive casting tool by a housing that forms part of the subsequent electroacoustic transducer. The solution in accordance with the invention thus makes it possible to produce electroacoustic transducers of the type mentioned above both in small production runs and individually at low cost; they can be manufactured virtually without any tools, but the piezoelectric elements are fixed and held tightly and fixedly in them by a hardening casting compound. However, the casting compound can also consist of a gel, a high-voltage oil, or a sand-like powder suspended in a liquid. This results in a composite structure between the piezoelectric elements, the bearer(s), and the housing, which forms a stable whole. Such a housing, which can be manufactured, for example, by 3D printing, makes it possible to vary the transducer in its external shape and in its internal design more or less at will, without the need for expensive tool changes. In accordance with the invention, the housing thus replaces the casting tool and has the additional advantage that it does not even have to be removed after the casting compound has hardened. It has the further advantage that casting compounds can also be used that do not harden, but remain liquid or pasty. If the housing does not have a load-bearing function, as is the case in a multiplicity of embodiments, it can be manufactured cost-effectively and, if required, also in individual made-to-order production.

In accordance with a development of the invention, the free space is preferably in-filled with a hardening plastic. Such a hardening plastic can be, for example, a thermoplastic, which hardens after cooling within the housing, or a thermoset, which hardens chemically, similar to the casting compounds known from the prior art. Such a plastic can advantageously be cross-linked with fibers that increase its strength, for example, glass fibers, carbon fibers, particles, or other macroscopic bodies.

In accordance with a further development of the invention, the housing is advantageously configured such that it has at least one opening for in-filling the casting compound, and at least one other opening for the escape of gas when in-filling the casting compound. Needless to say, depending on the design, a plurality of in-filling and degassing openings can also be provided in the housing. Here the opening for the escape of gas can aid the in-filling process by the provision of evacuation in this region. In the case of a hardening plastic, these openings can remain open after the free space has been in-filled with the casting compound and hardened, but in the case of casting compounds which remain in a liquid, pasty or hybrid form, these openings can be provided such that they can be closed, either by providing an appropriate in-filling valve in the in-filling opening and a plug for the outlet opening, or by a specific closure of these openings at the end of production, either by welding, adhesive bonding, or the like.

The housing is advantageously constructed in a number of parts, and consists of at least two housing parts, which are fixedly connected to each other after incorporation of the bearer or bearers with the piezoelectric elements. This preferably takes place by means of material bonding, whether by adhesive bonding, welding or the like.

In accordance with the invention, the housing is advantageously configured as a closed housing, that is to say, as a housing that completely replaces the casting tool and securely accommodates the casting compound. However, in accordance with an alternative form of embodiment of the invention, provision can be made for the housing to be partially open, that is to say, not completely closed. In this case, the open side is provided on the side facing away from the coupling face, that is to say, on the rear side of the housing. Here, depending on the design of the transducer, a housing wall can be dispensed with if, for example, a planar transducer is to be cast with a hardening plastic, since no casting tool is then required in this region. In the context of the present invention, therefore, a partially open housing is only to be understood as such a housing that is open on one side on which no tool is required when in-filling the free space with the casting compound; this can typically be the case on the rear side in particular designs.

It can be advantageous to design both the housing and the bearer(s) in plastic, these can be manufactured by 3D printing, or can also be configured as injection molded parts if larger quantities are to be manufactured. In particular, parts of the housing can also advantageously consist of metal, but so can the bearer or bearers, in which case special precautions usually have to be taken with regard to electrical insulation. The coupling face advantageously consists of metal, especially if a liquid-filled supply line connects here, in order to prevent moisture from diffusing through the housing into the casting compound. In order to increase the strength of the components, it can be advantageous to use composite materials here, in particular also in the region of the bearer(s), which form essential parts of the later composite. Even in the case of configurations in which the casting compound is non-hardening, the bearer forms the essential mechanical composite between the piezoelectric elements. The bearer(s) is/are then suitably fixed to the housing in a form fit, a force fit and/or material bonding.

In particular when using plastic, housing parts can advantageously be formed by deep drawing. This method is suitable for small and medium production runs. In order to ensure that no liquid diffuses from the coupling section into the transducer if a plastic housing is used, in accordance with an advantageous development of the invention the housing can be provided with a metal layer impervious to liquid, on at least the coupling face. Such a metal layer can be provided in the form of a foil, or can also be vapor deposited, and is preferably arranged on the inner face of the housing, since no special precautions then have to be provided to protect the metal layer. On the other hand, the metal layer can also be provided on the coupling face, but then at least a protective lacquer, or a further protective layer on top of the metal layer, will usually have to be provided.

In particular in the case of transducers of smaller size, it is advantageous to provide the housing on the coupling face with a gel pad as a coupling section. Advantageously, such a gel pad can be replaced to enable different penetration depths, in particular in the case of self-focusing transducers.

In accordance with a further development of the invention, the housing can be configured such that an aperture is formed, preferably running through the front and rear faces, which is provided to accommodate a camera, an ultrasound transducer, or a sensor. Depending on the requirements, one or a plurality of such apertures can be provided. In this case, an aperture is advantageously completely closed off by the housing, so that, despite the aperture, the interior of the housing remains protected by the housing wall.

The inventive housing design makes it possible to manufacture individually adapted housings cost effectively, even in very small quantities. In this respect, the housing of the inventive transducer can advantageously be configured to be anatomically adapted to the specific application, at least on the coupling face, but not necessarily only on this side. If both the housing and the bearer are manufactured by 3D printing, then the shape of the housing and the arrangement of the piezoelectric elements in the latter can in practice be configured as required. For treatment of the extremities, for example, it is conceivable to design the housing as a tub shape, and to form the coupling face in terms of the floor of the tub. The piezoelectric elements can also be arranged within the housing such that a partially focused wavefront is created, as is useful for soft tissue treatment, for example.

For the arrangement of the piezoelectric elements within the bearer, it is advantageous if the bearer surrounds the piezoelectric elements circumferentially, at least in some sections, so that the piezoelectric elements are free on their front face and their rear side, and are thus accessible for electrical wiring. Piezoelectric elements on the same bearer plane are typically connected in parallel, where-by the connections of successive bearer planes are led out separately in order to be able to control the time sequence of the voltage application, and thus the propagation of the wave fronts.

Depending on the design of the sound transducer, the piezoelectric elements can be activated individually, in groups, or all together. The electrical wiring must be carried out accordingly. A large variance of the activation can be achieved by individual activation, which, however, requires correspondingly complex wiring. With individual or group activation, activation is possible in a time variance, whereby practically any wave fronts can be generated.

The inventive electroacoustic transducer is typically manufactured by first arranging the piezoelectric elements in one or a plurality of bearers. A bearer is a component that has a multiplicity of recesses, in each of which a piezoelectric element can be clamped. Expediently, the electrical wiring of the elements is then carried out, after which the resulting structure is incorporated into the typically multi-part housing, which is then fixedly and tightly sealed by adhesive bonding or ultrasonic welding, by means of which the housing parts are joined together. The casting compound is then in-filled; this typically hardens after a predetermined time, after which the electroacoustic transducer is complete.

It should be noted that in addition to the aforementioned bearer that is provided with recesses, additional supporting bodies can also be provided within the housing. The housing itself can also form such a supporting body, in which case it is configured to be appropriately robust.

The inventive design of the electroacoustic transducer makes it possible to produce practically any shapes and structures. Thus, multi-curved transducers, segmented line transducers, transducers with stepped housings, as well as transducers with integrated individual domes and other special designs, can be manufactured at low cost, especially if the bearers and the housing are manufactured using the 3D printing process. The transducers in their entirety can also be provided with acoustic lenses, preferably arranged outside the housing, or the piezoelectric elements can preferably be provided with appropriate lenses inside the housing.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a cross-sectional view through a self-focusing transducer in accordance with the invention;

FIG. 2 is a linearly-focusing transducer constructed with two bearers with piezoelectric elements arranged one behind the other, as shown in FIG. 1;

FIG. 3 is a cross-sectional view through a transducer anatomically adapted for the foot; and

FIG. 4a, FIG. 4b FIG. 4c, and FIG. 4d are each a schematic representation of various types of transducer.

### DESCRIPTION OF PREFERRED EMBODIMENTS

Referring to the drawings, the electroacoustic transducer 1 shown in FIG. 1 takes the form of a self-focusing transducer, which has a housing 2, 3 consisting of plastic, which is configured in two parts, and consists of a first housing part 2 forming the coupling face of the transducer 1; this is configured in the shape of a cup, has a concave shape directed towards the coupling face, and in other respects is cylindrical. The cylindrical part of the housing is closed off by a rear housing part 3, which also consists of plastic, and is fixedly and tightly connected to the housing part 2 by means of ultrasonic welding. Inside the housing 2, 3 is arranged a bearer 4, in which a multiplicity of piezoelectric elements 5 are arranged. The piezoelectric elements 5 have an essentially cylindrical shape, they are fixed peripherally in recesses of the bearer 4 in a form fit and a force fit, and are electrically wired up on their end faces. They are arranged within the dome-shaped bearer 4 such that, when the elements 5 are simultaneously electrically activated, an acoustic pressure wave is generated, the focus 6 of which lies at some distance from the coupling face opposite the concave housing part 2.

The bearer 4, with the piezoelectric elements 5 arranged in the latter, is fixed in a form fit within the housing 2, 3 by two cylindrical rings 7, which with their outer face abut against the inner side of the housing 2, 3, and with one end face abut against the front inner face of the housing part 2, or against the rear inner face of the housing part 3. The bearer 4 is integrated in a form fit between the other end faces of the rings 7. Ports 8, 9 are provided circumferentially in the cylindrical region in the front housing part 2, wherein port 8 is an opening for the in-filling of a casting compound into the housing 2, 3, and port 9 is provided for the connection of a vacuum line, or as an outlet for the air that is escaping as the casting compound is in-filled into the housing 2, 3. The casting compound is thus in-filled into the housing 2, 3 via the port 8, the in-filling process can be aided by the connection of a vacuum pump to the port 9, the degassing takes place at least via the port 9, that is to say, the gas volume displaced by the in-filled casting compound 10 escapes through the said port 9.

The casting compound 10 is in-filled under pressure through the port 8 into the housing 2, 3 after the bearer 4 with the piezoelectric elements 5 arranged therein has been fixed between the rings 7 inside the housing 2, 3 and the housing parts 2, 3 have been fixedly and tightly connected to each other by adhesive bonding or welding. Here the electrical connections of the piezoelectric elements 5 are already wired up, and the corresponding connection leads 15 are tightly led out of the housing 2, 3 on the rear side. After the casting compound 10 has hardened, the ports 8, 9 are sealed by the hard casting compound 10 located therein. In other respects, the casting compound has formed a solid coherent body, which can now be installed in an appropriate medical device, wherein the concave outer side of the housing part 2 forms the coupling face, to which a correspondingly shaped gel body (not shown) is detachably attached. Alternatively, a fluid-filled coupling section with a membrane can here be provided, in which case the housing part 2 is preferably provided with a metal layer impervious to diffusion on the inner face in order to prevent fluid from penetrating into the transducer 1.

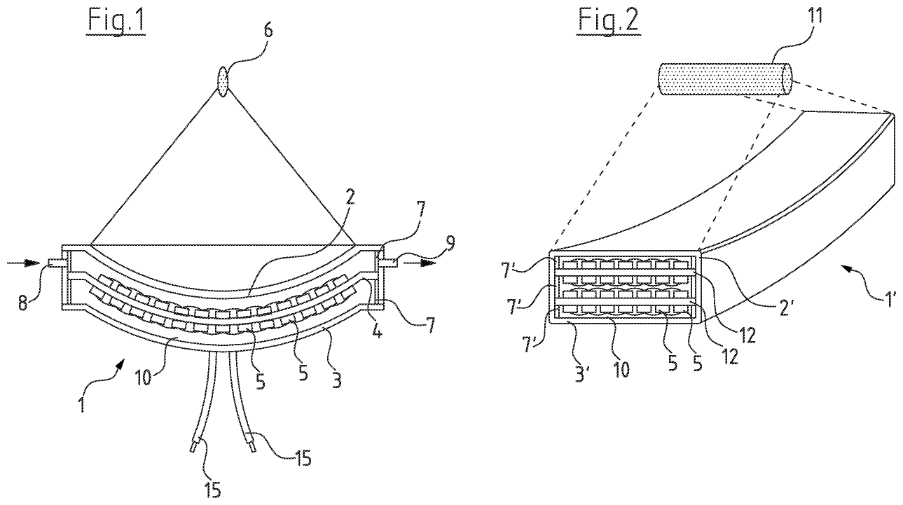

In the electroacoustic transducer 1′ shown in FIG. 2, the transducer housing 2′, 3′ also consists of two housing parts, namely a front housing part 2′ facing towards the coupling face, and a rear housing part 3′. The housing 2′/3′ shown in FIG. 2 has the shape of a cylindrical ring section and is intended for a linearly-focusing transducer 1′. Two bearers 12 are arranged within the housing, each of which has a multiplicity of piezoelectric elements 5, each of which is wired up in parallel on the bearer face, but is led out separately on the rear side via connecting leads (not shown). In this embodiment also, the bearers 12 are arranged in a form fit within the housing 2′, 3′ by means of rings 7′ that are essentially rectangular in plan view. The arrangement is such that the piezoelectric elements 5 focus the generated sound wave in a linear manner; the focal region is marked as 11 in FIG. 2. In other respects, the transducer 1′ shown in FIG. 2 is constructed in the same way as that shown in FIG. 1. The two bearers 12 with piezoelectric elements 5 incorporated therein are fixedly connected to each other and to the surrounding housing 2′, 3′ by a casting compound 10. This housing 2′, 3′ thus replaces the casting tool during the casting process. The housing parts 2′, 3′ themselves, as well as the bearers, can be manufactured cost-effectively, for example using 3D printing, so that individual housing shapes can also be implemented economically in individual made-to-order production.

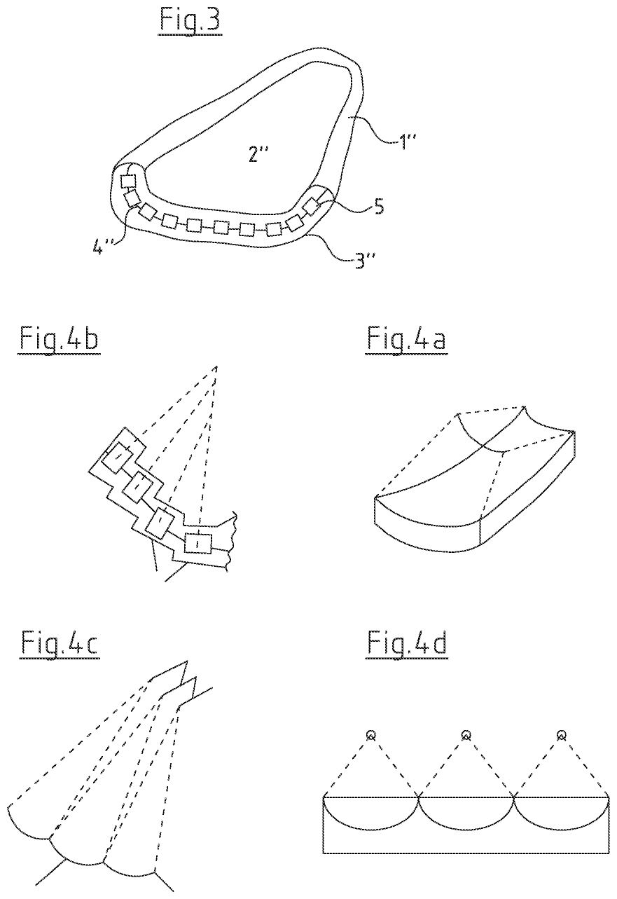

FIG. 3 shows schematically how an anatomically adapted transducer 1″ can be configured. The transducer 1″ shown there is adapted to the lower shape of a foot. In the edge regions in which the transducer 1″ is of curved design to encompass the side of the foot, the piezoelectric elements 5 located therein are provided with acoustic dispersion lenses (not shown). By this means any unwanted focusing in this region is prevented. In an analogous manner, the opposite effect can be achieved by an arrangement of collection lenses. These acoustic lenses are expediently provided on the housing, and can be provided on the inner face of the upper housing part 2″, but can also be provided on the outer face, or on both faces. In this embodiment variant, the piezoelectric elements 5 are again integrated into a bearer 4″, which is held in a form fit within the housing formed by the housing parts 2″ and 3″. The free space between the piezoelectric elements 5 and the housing 2″, 3″ is completely in-filled with a hardening plastic.

FIGS. 4a to 4d show examples of other electroacoustic transducers to illustrate the variety of housing shapes that can be implemented with the present design. FIG. 4a shows a bi-axially curved housing in which the piezoelectric elements, following the shape of the housing, generate a focus that corresponds to a curved line.

In FIG. 4b, the transducer is arranged in steps to achieve a spread in the depth of the individual piezoelectric elements, and thus a defocusing.

In the arrangement shown in FIG. 4c, a plurality of linearly-focusing transducers are arranged in a common housing.

The embodiment in FIG. 4d shows a transducer with a plurality of adjacent point-form focusing sections.

The electroacoustic transducers described above are configured exclusively for medical applications and are used to generate shock waves such as are used in the treatment of the human and animal body in medicine. For the coupling of the transducer to the body, a liquid-filled or gel-filled initial section is typically provided, for example in the form of a gel pad, such as contributes to the prior art.

While specific embodiments of the invention have been shown and described in detail to illustrate the application of the principles of the invention, it will be understood that the invention may be embodied otherwise without departing from such principles.

### LIST OF REFERENCE NUMBERS

1, 1′, 1″ Electroacoustic transducer
2, 2′ Front housing part
3, 3′ Rear housing part
4, 4″ Bearer
5 Piezoelectric elements
6 Focus
7, 7′ Rings
8 Input port
9 Output port
10 Casting compound
11 Focal region
12 Bearer in FIG. 2
15 Connection lead

1. An electroacoustic transducer for the generation of shock waves for the treatment of the human or animal body, the electroacoustic transducer comprising:
a housing;
at least one bearer;
piezoelectric elements, which are arranged in the at least one bearer within the housing; and,
a casting compound, wherein a free space formed between the piezoelectric elements and the housing is in-filled with the casting compound.

2. The electroacoustic transducer according to claim 1, wherein the free space is in-filled with a hardening plastic as the casting compound.

3. The electroacoustic transducer according to claim 1, in which wherein the housing has at least one opening for the in-filling of the casting compound, and at least one other opening for the escape of gas when in-filling the casting compound.

4. The electroacoustic transducer according to claim 1, wherein the housing is formed from at least two housing parts.

5. The electroacoustic transducer according to one claim 1, wherein the housing is configured as a closed housing.

6. The electroacoustic transducer according to claim 1, wherein the housing part is configured to be partially open, with an open side, wherein the open side is a rear side of the housing arranged opposite, facing away from, a coupling face.

7. The electroacoustic transducer according to claim 1, wherein the housing and/or the bearer are formed from plastic, metal, or a composite material.

8. The electroacoustic transducer according to claim 1, wherein at least one housing part is formed by deep drawing, injection molding, or with the 3D printing method.

9. The electroacoustic transducer according to claim 1, wherein the housing, at least on a coupling face, is provided on an inner face with a metal layer impervious to liquid.

10. The electroacoustic transducer according to claim 1, wherein the housing is provided on a coupling face with a gel pad as a coupling section.

11. The electroacoustic transducer according to claim 1, wherein the housing has at least one aperture, for the accommodation of a camera, an ultrasound transducer, a sensor, or the like.

12. The electroacoustic transducer according to claim 1, wherein the housing, at least on a coupling face, is configured so as to be anatomically adapted to a specific application.

13. The electroacoustic transducer according to claim 1, wherein the bearer circumferentially surrounds the piezoelectric elements, at least in some sections, and the piezoelectric elements are electrically connected on a respective front face of the piezoelectric elements and on a respective rear side of the piezoelectric elements.

14. The electroacoustic transducer according to claim 1, wherein the piezoelectric elements arranged in the bearer can be electrically activated individually, in groups, or all together.

15. A method for the manufacture of a transducer, the method comprising the steps of:
arranging piezoelectric elements and electrically connecting piezoelectric elements in at least one bearer, wherein the at least one bearer, populated with piezoelectric elements, is arranged in a housing
in-filling a free space formed between the piezoelectric elements, the bearer and the housing with a casting compound.

16. The electroacoustic transducer according to claim 4, wherein the at least two housing parts are connected to one another by means of material bonding.

17. The electroacoustic transducer according claim 1, wherein the at least one aperture extends through a front side and a rear side of the housing.
